(19) United States
(12) Patent Application Publication
(10) Pub. No.: US 2017/0173270 A1
(43) Pub. Date: Jun. 22, 2017

(54) Self-Injection Tool with Movable Needle Shroud

(71) Applicant: AbbVie Inc., North Chicago, IL (US)

(72) Inventors: Kei NAKAMURA, Tokyo (JP); Junichi YOSHIDA, Chiba (JP)

(73) Assignee: AbbVie Inc., North Chicago, IL (US)

(21) Appl. No.: 15/118,314

(22) PCT Filed: Feb. 12, 2014
(86) PCT No.: PCT/US2014/016021

§ 371 Date: Aug. 11, 2016

(51) Int. Cl. A61M 5/32

(52) U.S. Cl. CPC A61M 5/3257; A61M 5/3202

(57) ABSTRACT

Self-injection tool includes a base and a shroud member. The base includes a proximal end, a distal end, and a barrel engagement portion. The barrel engagement portion defines a cavity to receive at least a portion a syringe barrel such that the distal end of a needle extends a first distance from the distal end of the base. The shroud member includes a proximal end and a distal end. The proximal end is movably coupled to the base. The shroud member is movable between a fully-extended position and a fully-retracted position. In the fully-extended position the shroud member is an extended distance from the distal end of the base. In the fully-retracted position the shroud member is a retracted distance from the distal end of the base. The extended distance is greater or equal to the first distance; the retracted distance is less than the first distance.

### BACKGROUND

Field of Disclosed Subject Matter

The present disclosed subject matter relates to a self-injection tool for injecting a substance, such as a therapeutic agent, into a patient.

Description of Related Art

Administration of therapeutic agents, such as pharmaceutical or biologic agents, can be performed by injection, which can include for example, subcutaneous or intramuscular injection. An injection device, such as a syringe or other container containing a therapeutic agent in fluid communication with a needle or jet, can be manually operated by a user to perform the injection. Injections can often be performed by trained medical personnel. Alternatively, a patient can be trained to use an injection device for self-injection. Moreover, injection device containers, such as a syringe, can be pre-filled with a therapeutic agent for patient use, to prevent or inhibit contamination of the therapeutic agent as well as avoid the need for the patient to fill the container. However, self-injection can be difficult for some patients, for example due to limited dexterity or a fear of needles.

Self-injection tools can aid patients in performing self-injections, as well as assisting in performing injections by a health care provider or the like. Self-injection tools can be used, for example, to assist with operating a manual injection device, such as a standard pre-filled syringe. Such tools can be configured, for example, to receive standard pre-filled syringes and release the syringes after use, and thus allow for repeated use of the self-injection tool by the patient. Self-injection tools can include a number of features to assist the patient to operate the manual injection device, such as removing a needle cap from the needle of a pre-filled syringe and guiding the insertion of the needle into the patient. Various types of self-injection tools have been described, for example in Japanese Patent No. 3,143,302 and Japanese Patent Application Publication No. 2011-98133, each of which is incorporated by reference herein in its entirety.

However, there remains a need for further improvement of known self-injection tools. For example, it can be desirable to have a self-injection tool that can be comfortable and easy for patients to manipulate, as well as conceal the needle from the patient's view

### SUMMARY

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

To achieve these and other advantages and in accordance with the purpose of the disclosed subject matter, as embodied and broadly described, the disclosed subject matter includes a self-injection tool for injecting a beneficial agent from a syringe. The syringe includes a barrel, a shoulder, a needle, a flange, a cap, and a plunger. The barrel includes a first end and a second end. The shoulder is proximate the first end. The needle is in fluid communication with the barrel and includes a proximal end and a distal end extending from the first end of the barrel. The flange is disposed proximate the second end of the barrel. The cap covers the needle and includes an end disposed proximate the shoulder. The plunger is movable within the barrel to expel the beneficial agent through the needle. The self-injection tool includes a base and a shroud member. The base includes a proximal end, a distal end, and a barrel engagement portion. The barrel engagement portion defines a cavity having a longitudinal axis and is disposed between the proximal and distal end of the base to receive at least a portion of the barrel with the distal end of the needle extending a first distance from the distal end of the base. The shroud member includes a proximal end and a distal end. The proximal end of the shroud member is movably coupled to the base, the shroud member being movable between a fully-extended position wherein the distal end of the shroud member is an extended distance from the distal end of the base and a fully-retracted position wherein the distal end of the shroud member is a retracted distance from the distal end of the base. The extended distance is greater than or equal to the first distance, and the retracted distance is less than the first distance.

As embodied herein, the shroud member can be biased toward the extended position. The distal end of the shroud member can include a substantially planar engagement surface having a notch therein or the distal end of the shroud member can include a substantially planar engagement surface having a through-hole therein. The shroud member can include a tubular member disposed between the proximal end and the distal end of the shroud member.

For example, and as embodied herein, the base can further include at least one flange engagement portion defining a slot to receive at least a portion of the flange and inhibit movement of the barrel along the longitudinal axis. The base can include a housing, and in some embodiments, the self-injection tool can further include a cover. The cover can be hingedly joined to the base and rotatable relative the base between an open position and a closed position. The barrel engagement portion can be movable relative the housing. The barrel engagement portion can include a cap engagement portion. The cap engagement portion can have an opening sized to receive at least a portion of the first end of the barrel. The cap engagement portion can be positioned to engage the end of the cap proximate the shoulder and can be movable relative the housing to urge the cap away from the barrel. The cap engagement portion can include a U-shaped notch.

Furthermore, as embodied herein, the housing can include a bottom and at least one sidewall extending upwardly from the bottom. The cover can be joined to the housing proximate at least one of the at least one sidewall. The at least one sidewall can have a substantially rounded shape. The base can comprise a proximate face extending from the bottom and disposed perpendicularly to the at least one sidewall. The proximal face can include an opening to receive at least a portion of the plunger therethrough when the at least a portion of the barrel is disposed within the barrel engagement cavity.

Additionally, and as embodied herein, the cover can have one or more projections to engage the barrel when the cover is in the closed position. The cap engagement portion can be movable relative the at least one flange engagement portion when the cover is rotated relative the base from the open position to the closed position. The syringe can be held in engagement when the cover is moved to the closed position. The self-injection tool can further include a flange adaptor. The flange adaptor can include a recess to receive the flange and at least one wing element configured to engage the flange engagement portions.

Furthermore, as embodied herein, the base can further include a lock mechanism movable between a non-locking position and a locking position when the cover is rotated relative the base from the open position to the closed position. The proximal end of the shroud can include at least one locking projection. The locking projection can be configured to engage the lock mechanism when the lock mechanism is in the engaging position and the shroud is in the retracted position. The lock mechanism can be movable between the locking position and the non-locking position to disengage the at least one locking projection when the cover is rotated relative the base from the closed position toward the open position. Alternatively the cover can be configured to create a friction engagement between the barrel engagement portion and the cap engagement portion when the cover is in the closed position.

Additionally, as embodied herein, the beneficial agent can include a TNF inhibitor.

Furthermore, in accordance with the purpose of the disclosed subject matter, as embodied and broadly described, the disclosed subject matter includes a kit. The kit includes a syringe and a self-injection tool. The syringe includes a barrel, a shoulder, a needle, a flange, a cap, and a plunger. The barrel includes a first end and a second end and defines a longitudinal axis therebetween. The shoulder is proximate the first end. The needle is in fluid communication with the barrel and includes a proximal end and a distal end extending from the first end of the barrel and. The flange is disposed proximate the second end of the barrel. The cap covers the needle and includes an end disposed proximate the shoulder. The plunger is movable within the barrel to expel the beneficial agent through the needle. The self-injection tool includes a base and a shroud member. The base includes a proximal end, a distal end, and a barrel engagement portion. The barrel engagement portion defines a cavity disposed between the proximal and distal end of the base to receive at least a portion of the syringe barrel with the distal end of the needle extending a first distance from the distal end of the base. The shroud member includes a proximal end and a distal end. The proximal end of the shroud member is movably coupled to the base, the shroud member being movable between a fully-extended position wherein the distal end of the shroud member is an extended distance from the distal end of the base and a fully-retracted position wherein the distal end of the shroud member is a retracted distance from the distal end of the base. The extended distance is greater than or equal to the first distance, and the retracted distance is less than the first distance.

It is understood that both the foregoing general description and the following detailed description are exemplary and are intended to provide further explanation of the disclosed subject matter claimed.

The accompanying drawings, which are incorporated in and constitute part of this specification, are included to illustrate and provide a further understanding of the disclosed subject matter. Together with the description, the drawings serve to explain the principles of the disclosed subject matter.

### BRIEF DESCRIPTION OF THE DRAWINGS

FIG. 1 is a perspective view of the self-injection tool according to an illustrative embodiment of the disclosed subject matter.

FIG. 2 is a top view of the self-injection tool of FIG. 1.

FIG. 3 is a bottom view of the self-injection tool of FIG. 1.

FIG. 4 is a right side view of the self-injection tool of FIG. 1, the left side view being a mirror image of the right side view.

FIG. 5 is a front view of the self-injection tool of FIG. 1.

FIG. 6 is a rear view of the self-injection tool of FIG. 1.

FIG. 7 is a perspective view of an exemplary shroud member of the self-injection tool of FIG. 1.

FIG. 8 is a perspective view of an exemplary housing of the self-injection tool of FIG. 1.

FIG. 9 is a perspective view of an exemplary cover of the self-injection tool of FIG. 1.

FIG. 10 is a perspective view of an exemplary syringe engagement element of the self-injection tool of FIG. 1.

FIG. 11 is a perspective view of an exemplary lock mechanism of the self-injection tool of FIG. 1.

FIG. 12 is a perspective view of an exemplary back cover of the self-injection tool of FIG. 1.

FIG. 13 is a plan view of the back cover of FIG. 12.

FIG. 14 is a perspective view of an exemplary insert of the self-injection tool of FIG. 1.

FIG. 15 is a perspective view of an exemplary insert of the self-injection tool of FIG. 1.

FIG. 16 is an exploded view of the various components of the illustrative self-injection tool of FIG. 1.

FIG. 17 is a bottom view of the self-injection tool of FIG. 1, with the cover in an open position and the shroud member in a fully-extended position.

FIG. 18 is a bottom view of the self-injection tool of FIG. 1, with the cover in a closed position and the shroud member in a fully-extended position.

FIG. 19 is a bottom view of the self-injection tool of FIG. 1, with the cover in a closed position and the shroud member in a fully-retracted position.

FIG. 20 is a top perspective view of the self-injection tool of FIG. 1, with the cover in an open position.

FIG. 21 is a top view of the self-injection tool of FIG. 1, with the cover in a closed position and depicted in dotted lines for clarity.

FIG. 22 is a perspective view of an exemplary syringe for use with the self-injection tool of FIG. 1.

FIG. 23 is a perspective view of the self-injection tool of FIG. 1, with the cover in the open position and depicted in dotted lines for clarity, prior to insertion of the syringe into the self-injection tool.

FIG. 24 is a perspective view of the self-injection tool of FIG. 1, with the cover in the open position and depicted in dotted lines for clarity, and with the syringe inserted into the self-injection tool.

FIG. 25 is a perspective view of the self-injection tool of FIG. 1, with the cover in the closed position and depicted in dotted lines for clarity, and with the syringe inserted into the self-injection tool.

FIG. 26 is a perspective view of the self-injection tool of FIG. 1, with the cover in the closed position and depicted in dotted lines for clarity, the shroud member in the fully-retracted position.

### DETAILED DESCRIPTION OF THE PREFERRED EMBODIMENT

Reference will now be made in detail to the various exemplary embodiments of the disclosed subject matter, exemplary embodiments of which are illustrated in the accompanying drawings. The structure and corresponding method of operation of the disclosed subject matter will be described in conjunction with the detailed description of the system.

The apparatus and methods presented herein can be used for injecting any of a variety of suitable therapeutic agents or substances, such as a drug, into a patient. As used herein, a “self-injection tool” or “self-injection aid” or “tool” (used interchangeably herein) is intended to refer generally to a device to assist an individual (also referred to herein as a user, a patient or a health care provider) to administer a fluid substance, such as a therapeutic agent. In some embodiments, the tool can be configured to receive a syringe or other injection device that can be otherwise used without the self-injection tool. However, in alternate embodiments, the syringe or other injection device can be functional only with the use of the self-injection tool. The liquid beneficial agent can include a protein. In some embodiments, the liquid beneficial agent can include a TNF inhibitor or the like, such as adalimumab.

The syringe disclosed herein can include, for illustration and not limitation, a barrel, a shoulder, a needle, a flange, a cap, and a plunger. The barrel includes a first end and a second end. The shoulder is proximate the first end. The needle is in fluid communication with the barrel and includes a proximal end and a distal end extending from the first end of the barrel. The flange is disposed proximate the second end of the barrel. The cap covers the needle and includes an end disposed proximate the shoulder. The plunger is movable within the barrel to expel the beneficial agent through the needle.

In accordance with the disclosed subject matter herein, the self-injection tool for injecting a beneficial agent from a syringe generally includes a base and a shroud member. The base includes a proximal end, a distal end, and a barrel engagement portion. The barrel engagement portion defines a cavity having a longitudinal axis and is disposed between the proximal and distal end of the base to receive at least a portion of the syringe barrel with the distal end of the needle extending a first distance from the distal end of the base. The shroud member includes a proximal end and a distal end. The proximal end of the shroud member is movably coupled to the base, the shroud member being movable between a fully-extended position wherein the distal end of the shroud member is an extended distance from the distal end of the base and a fully-retracted position wherein the distal end of the shroud member is a retracted distance from the distal end of the base. The extended distance is greater than or equal to the first distance, and the retracted distance is less than the first distance.

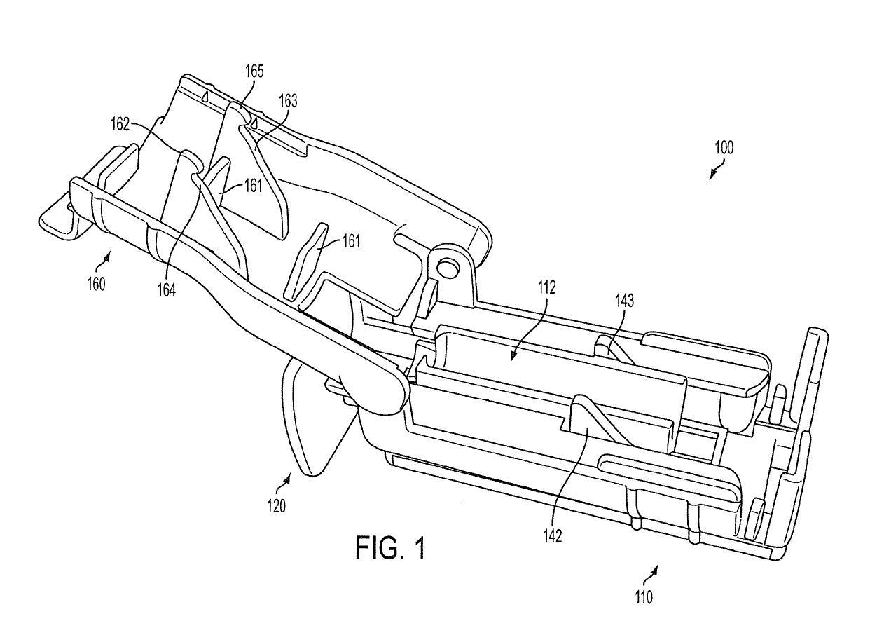

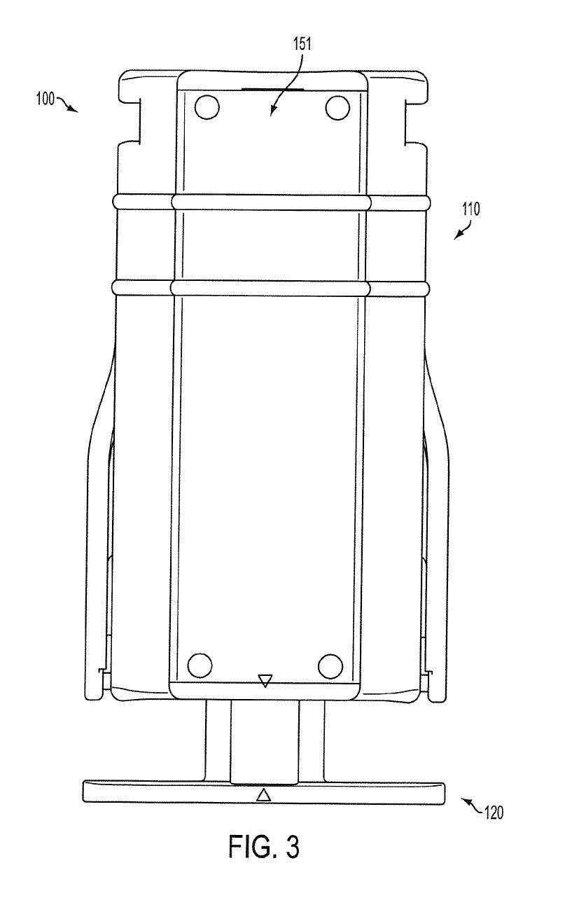

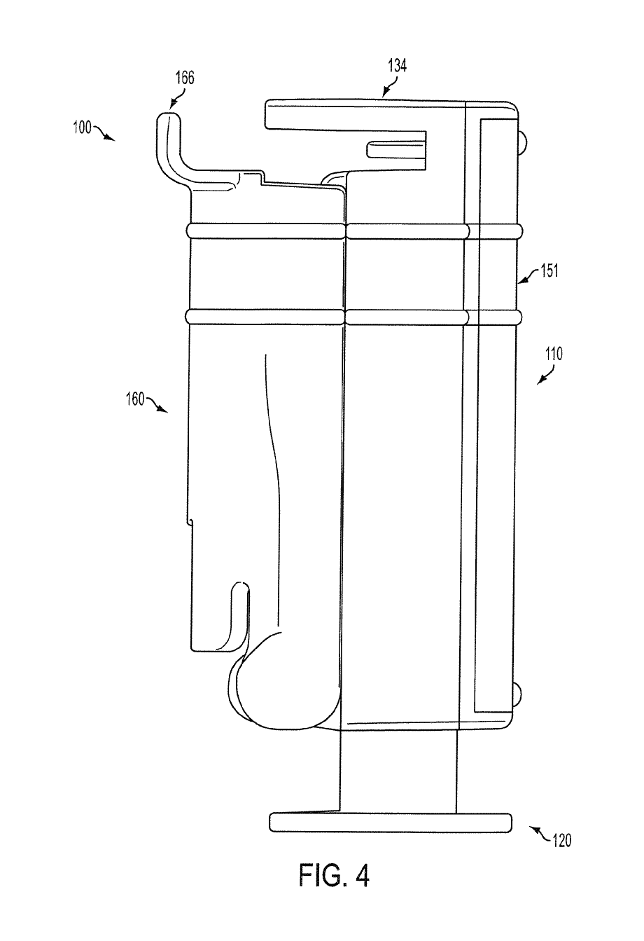

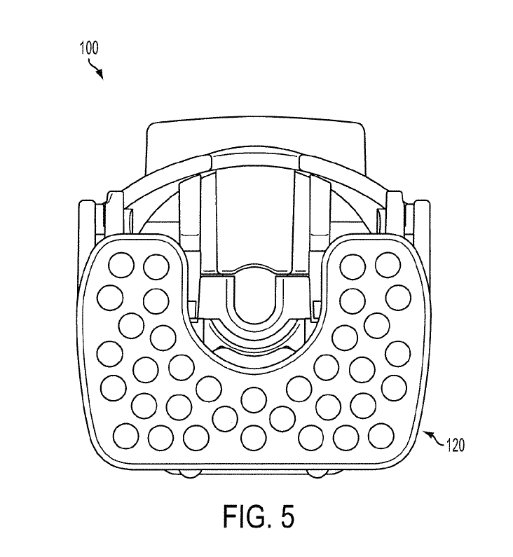

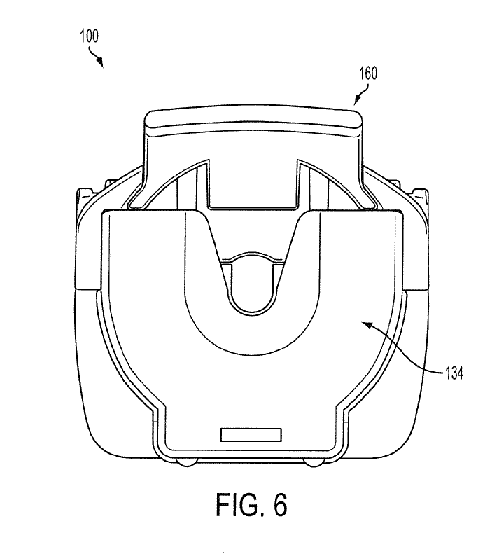

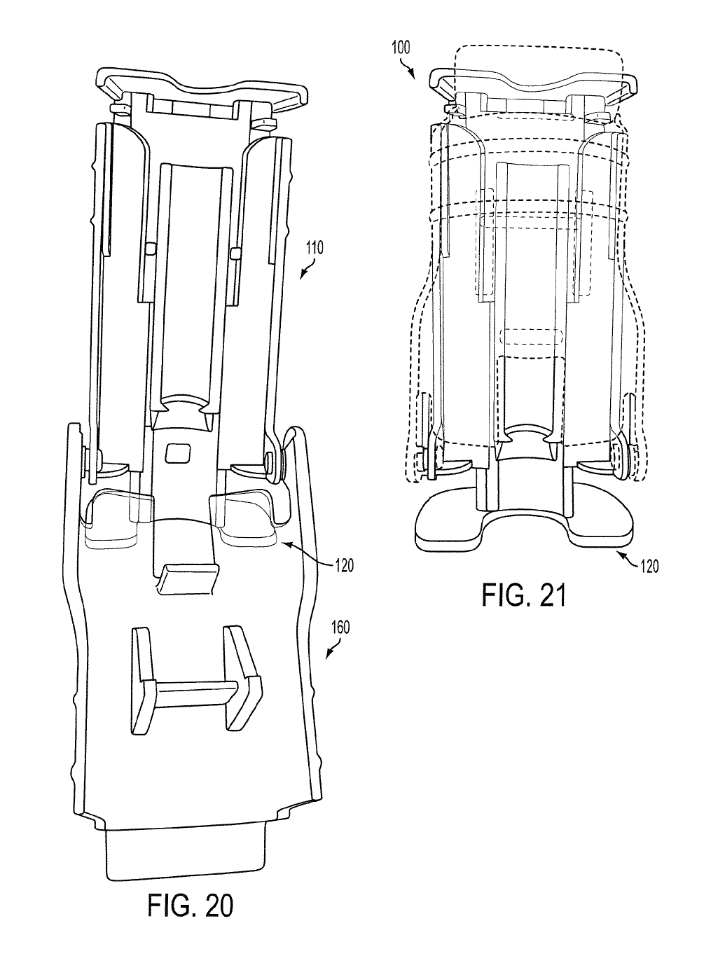

The accompanying figures, where like reference numerals refer to identical or functionally similar elements throughout the separate views, serve to further illustrate various embodiments and to explain various principles and advantages all in accordance with the disclosed subject matter. For purpose of explanation and illustration, and not limitation, exemplary embodiments of the self-injection tool are shown in FIGS. 1-25. While the present disclosed subject matter is described with respect to using the device to provide a subcutaneous injection of a TNF inhibitor or the like, such as adalimumab, one skilled in the art will recognize that the disclosed subject matter is not limited to the illustrative embodiment, and that the self-injection tool can be used to inject any suitable substance into a user, including any nutritional, pharmaceutical, or biological agents. In addition, the components and the method of using the self-injection tool are not limited to the illustrative embodiments described or depicted herein.

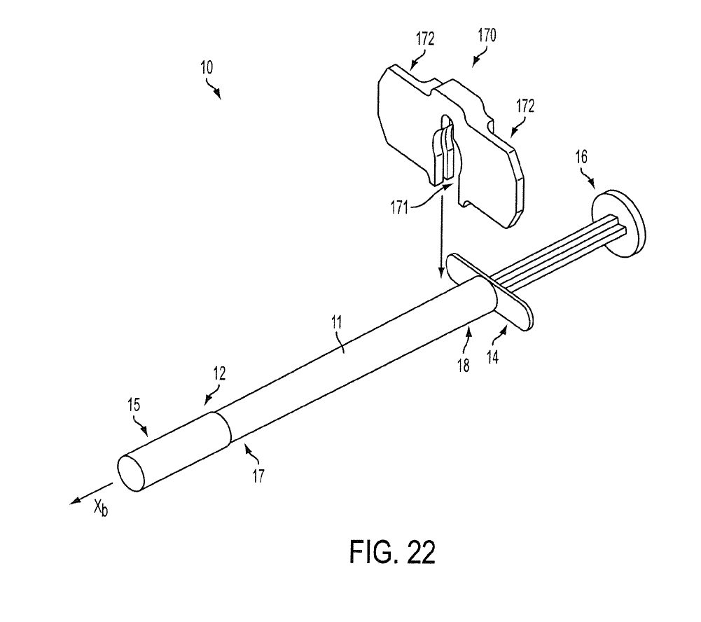

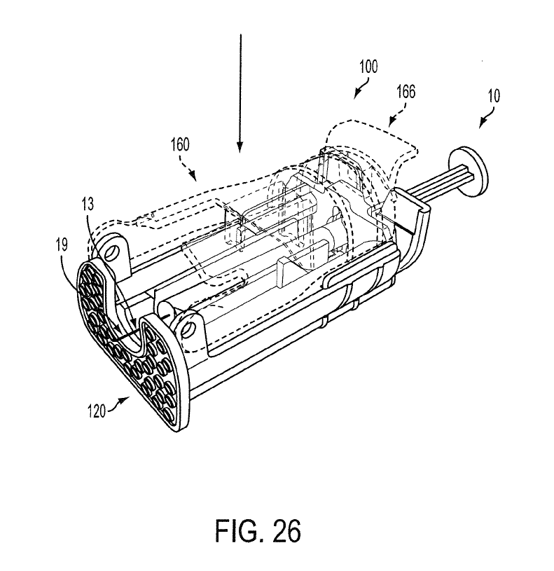

For the purpose of illustration and not limitation, with reference to FIG. 22, an exemplary syringe 10 for use with a self-injection tool according to the disclosed subject matter is shown. The syringe 10 can include a barrel 11 having a shoulder 12 proximate a first end 17 and a needle 13 (shown for example in FIG. 26) extending from the first end 17. A cap 15 can cover the needle 13 to prevent or inhibit inadvertent needle sticks and can include an end 21 disposed proximate the shoulder 12. The syringe 10 can include a flange 14 surrounding an opening proximate a second end 18. The needle 13 is in fluid communication with the barrel 11 and includes a proximal end 20 proximate from the first end 17 of the barrel 11 and a distal end 19 extending therefrom. The plunger 16 can be inserted into the opening proximate the second end 18 and movable within the barrel 11 to expel the beneficial agent through the needle 13.

According to the disclosed subject matter, a self-injection tool 100 is provided. With reference to FIGS. 1-6, an exemplary self-injection tool 100 includes a base 110, and a shroud member 120. The base 110 can include a barrel engagement portion 112. The barrel engagement portion 112 defines a cavity having a longitudinal axis x_{b} to receive at least a portion of the syringe barrel 11. The barrel engagement portion 112 is configured to receive the syringe barrel 11 such that the distal end 19 of the needle 13 extends a first distance D₁ from the distal end 111 of the base 110.

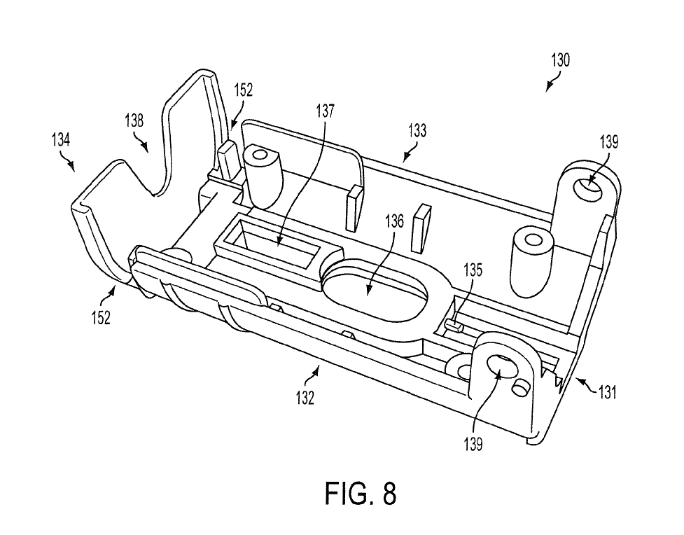

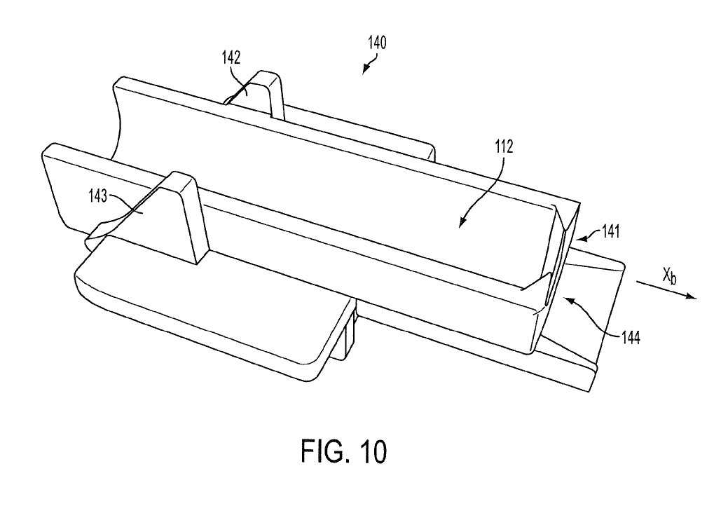

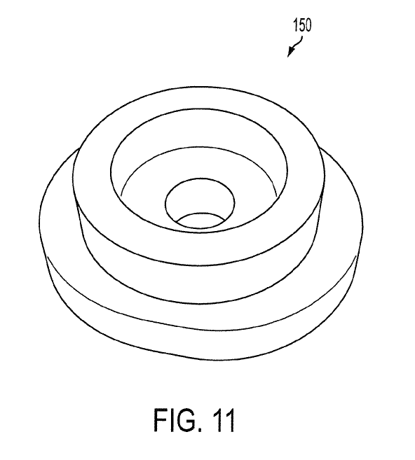

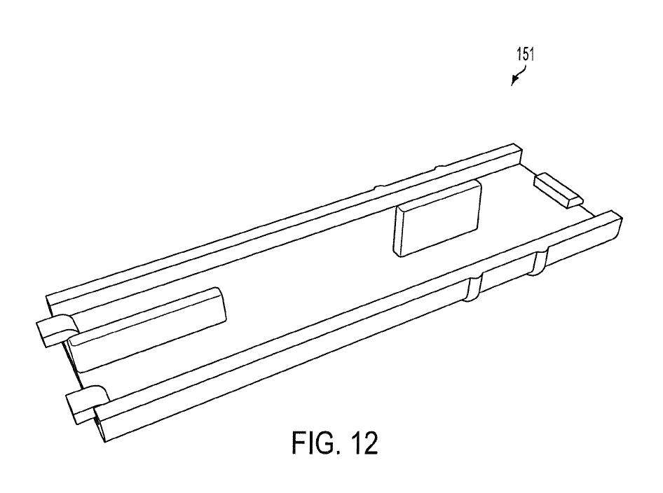

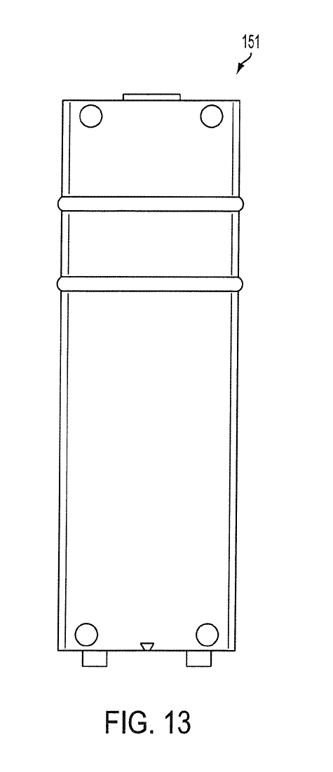

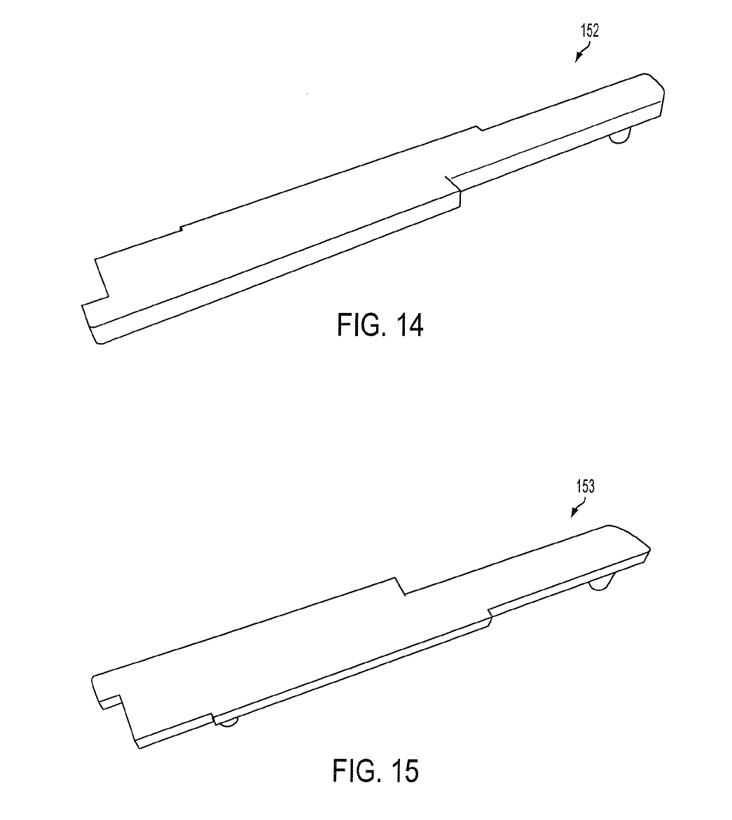

The base 110 can be configured as an assembly of various components. FIGS. 8, 10-15 show, for the purpose of illustration and not limitation, exemplary embodiments of components of base 110. Alternatively, base 110 can be configured as a unitary piece of material. The components of base 110 can generally include a housing 130 (FIG. 8) (which can include back cover 151 (FIGS. 12-13) and inserts 152, 153 (FIGS. 14-15, respectively)), a syringe engagement element 140 (FIG. 10), and a lock mechanism 150 (FIG. 11).

FIG. 8 shows an exemplary housing 130 of the base 110. As embodied herein, the housing 130 includes a bottom 131 and at least one sidewall 132, 133 extending from the bottom 131. Alternatively, the housing 130 can include a single sidewall or three or more sidewalls or sidewall portions. As shown, the sidewalls 132, 133 can have a substantially rounded shape, which can improve ergonomics to thus provide a user with improved comfort and ease of use when holding and/or manipulating the tool 100. Alternatively, the sidewalls can be substantially planar, or can have any other suitable shape. The bottom 131 can also include bias-engaging member 135, which can be configured, for example, as a protrusion sized to engage biasing member 23 (shown for example, in FIG. 19). Furthermore, and as embodied herein, bottom 131 can include a biasing slot 137 sized to receive biasing member 24 (shown for example, in FIG. 19) and an engagement slot 136 sized to receive lock mechanism 150, as described further herein.

Additionally, and as embodied herein, the housing 130 can include a proximal face 134 extending from the bottom 131. The proximal face 134 can also include a notch or opening 138. The opening 138 can be sized to receive at least a portion of the plunger 16 therethrough when at least a portion of the barrel 11 is disposed within the barrel engagement cavity 112, as described further herein. The housing 130 also includes cover-receiving holes 139 sized to receive a cover 160, as described further herein.

In some embodiments, the base 110 can further include one or more flange engagement portions 152. For example, and not limitation the flange engagement portions 152 can define one or more slots to receive at least a portion of the flange 14 of the syringe 10. The flange engagement portions 152 can thus restrict or inhibit movement of the syringe barrel 11 along longitudinal axis x_{b}, as described further below.

Referring now to FIG. 22, for the purpose of illustration and not limitation, in some embodiments, the self-injection tool 100 can further include a flange adaptor 170. The flange adaptor 170 can include a recess 171 sized to receive the flange 14 of the syringe 10. The flange adaptor 170 can also include at least one wing element 172 sized to be received within and engage the flange engagement portions 152 such that the recess 171, and thus the syringe barrel 11, is substantially in alignment with the barrel engagement portion 112.

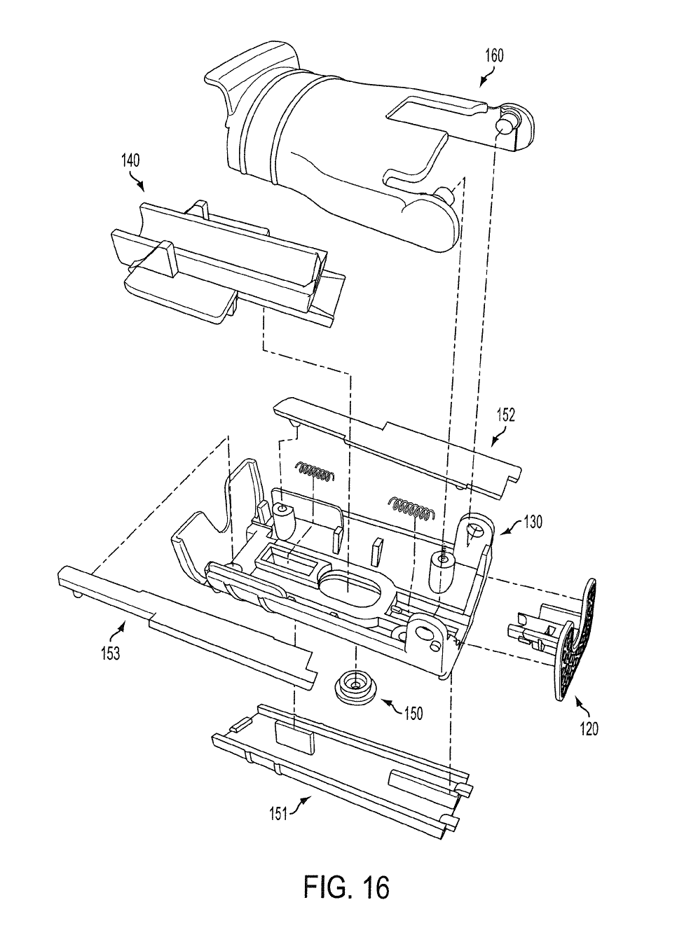

As noted above, the base 110 further includes a barrel engagement portion 112. The barrel engagement portion 112 can be fixed in position, such as integral with the remainder of the base. Alternatively, and as embodied herein, the barrel engagement portion 112 can be moveable, such as to facilitate removal of the syringe cap. For example, the barrel engagement portion 112 can include a syringe engagement element 140 is shown for purpose of illustration and not limitation in FIG. 10. As embodied herein, the syringe engagement element 140 can include the barrel engagement cavity sized to receive the barrel 11 of the syringe 10 and defining a longitudinal axis x_{b}. The syringe engagement element 140 can also include a cap engagement portion 141 and cover engagement portions 142, 143. The cap engagement portion 141 can include a notch 144, which can have a U-shape or other suitable shape, to define an opening sized to receive at least a portion of the first end 17 of the barrel 11, proximate the shoulder 12. With reference to FIG. 16, as embodied herein, syringe engagement element 140 can be coupled to a lock mechanism 150 through slot 136. Syringe engagement element 140 can thus be moveable longitudinally along slot 136 between a first position and a second position. Accordingly, the cap engagement portion 141 can be movable relative the one or more flange engagement portions 152 along the longitudinal axis x_{b} to urge the cap 15 away from the barrel 11, as discussed further herein. As embodied herein, biasing element 24 (shown for example in FIG. 19) disposed within biasing slot 137, engages and biases the syringe engagement element 140 towards the first position. Biasing element 24 can be configured, for example and without limitation, as a mechanical spring, or any other suitable biasing element.

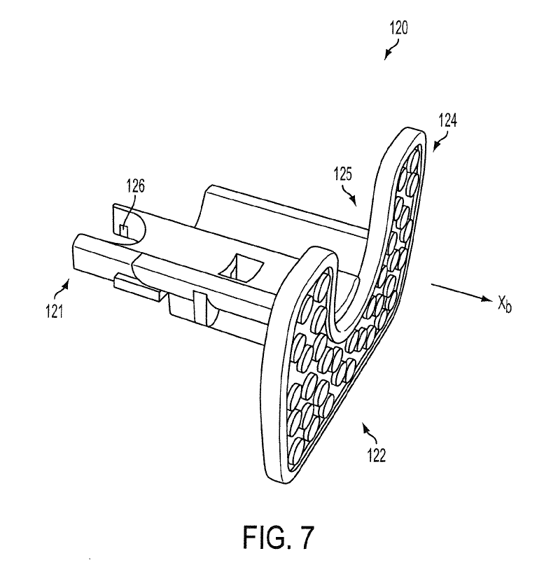

Referring now to FIG. 7, for purpose of illustration and not limitation, an exemplary shroud member 120 includes a proximal end 121 and a distal end 122. The shroud member 120 can be joined to base 110 and movable relative base 110 along the longitudinal axis x_{b} between a fully-extended position and a fully-retracted position. In the fully-extended position, shown for example in FIGS. 1, 17, and 18, the distal end 122 of the shroud member 120 extends an extended distance D₂ from the distal end 111 of the base 110. In the fully-retracted position, shown for example in FIG. 19, the distal end 122 of the shroud member 120 extends a retracted distance D₃ from the distal end 111 of the base 110. As embodied here, the extended distance D₂ is at least equal to or greater than the first distance D₁. The retracted distance D₃ is less than the first distance D₁. As such, for example and as embodied herein, when shroud member 120 is in the extended position, shroud 120 at least partially covers needle 13 of a syringe 10 engaging the syringe engagement member 140. The shroud member 120 therefore can prevent or inhibit inadvertent needle sticks and can conceal the needle 13 from view by the user. When the shroud member 120 is in the retracted position, the distal end 19 of the needle 13 can be exposed, for example to allow insertion of the needle 13 into an injection site.

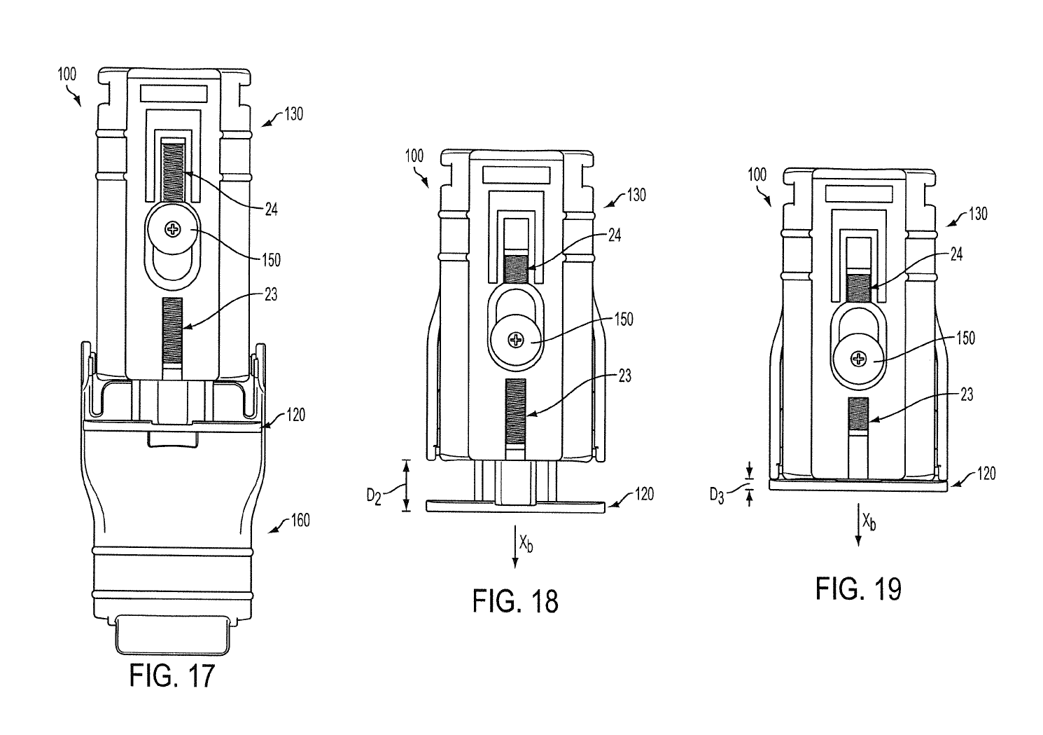

As embodied herein, the shroud member 120 can be biased toward the extended position. With reference to FIGS. 17-19, shroud member 120 can include a bias-engaging member (not shown) to engage a biasing member 23. The biasing member 23 can be configured, for example and without limitation, as a mechanical spring, or any other suitable biasing element. The distal end of the shroud member 120 can include a substantially planar engagement surface 124. The engagement surface 124 can be sized and shaped to engage an injection site, such as the user's skin during injection. In some embodiments, the engagement surface 124 can include one or more surface features, such as textured ribs or dimples, which can increase friction proximate the engagement surface 124 and thus reduce or prevent unwanted slipping of the engagement surface 124 along the injection site. The engagement surface 124 can have a notch 125 therein aligned with the needle 13 when the barrel 11 of the syringe 10 is received within the barrel engagement portion 112. Alternatively, the planar engagement surface 124 can have a through-hole, rather than a notch 125. As a further alternative, the shroud member 120 can include a tubular portion disposed between the proximal end 121 and the distal end 122. The tubular portion can surround the needle 13 when disposed within the barrel engagement portion 112 to further conceal the needle 13 from view when the shroud 120 is in the extended position.

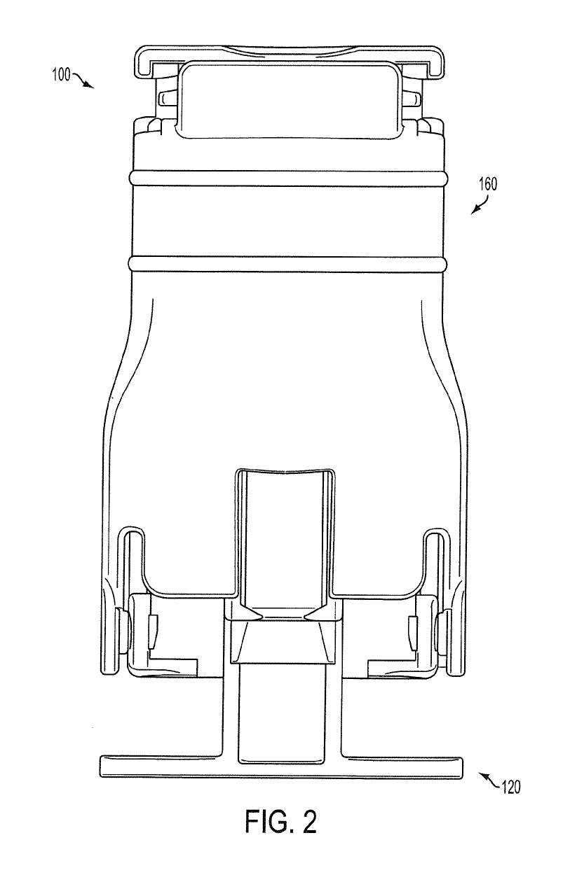

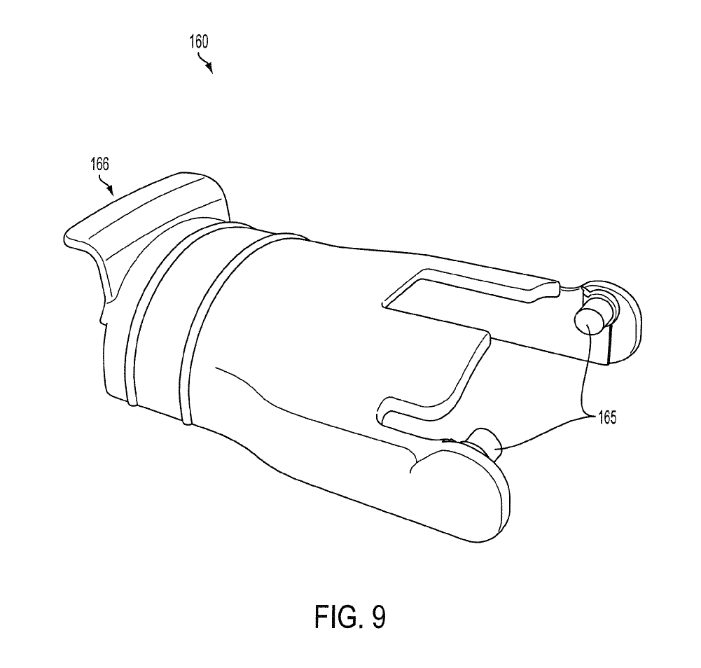

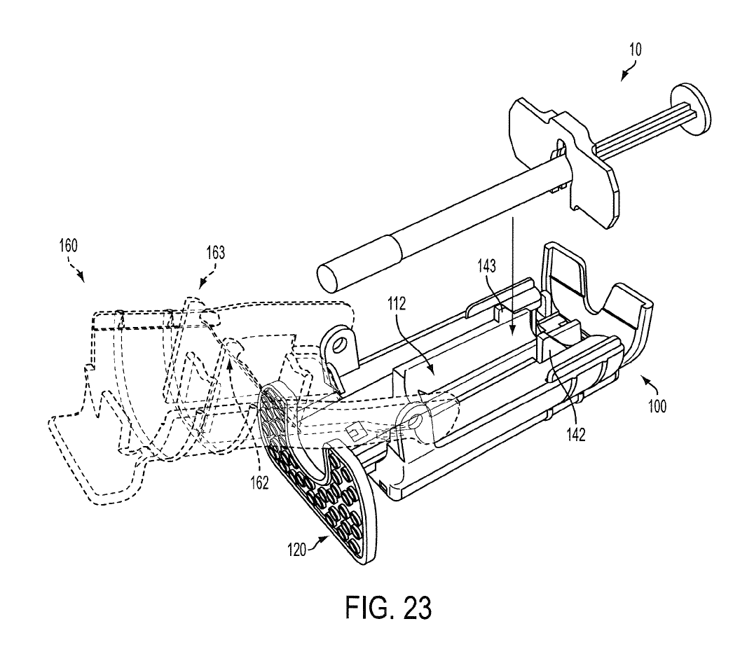

With reference to FIG. 9, the self-injection tool 100 can include a cover 160. The cover 160 can include grip member 166. The cover 160 can be hingedly joined to the base 110 such that the cover 160 is rotatable between an open position (shown for example in FIGS. 1 and 23) and a closed position (shown for example in FIGS. 2-6, and 25). As shown for example in FIGS. 1-2, the cover 160 can be coupled to the base via cover-receiving holes 139 to receive one or more protrusions 165 of the cover 160. The cover can be opaque to further conceal the needle from view, or transparent to allow visibility of the contents of the syringe.

With reference to FIG. 1, the cover can also include one or more projections 161 sized to engage the barrel 11 and prevent or inhibit movement of the barrel 11 away from the barrel engagement portion 112 when the cover 160 is in the closed position. The cover 160 can also include base engagement members 162, 163. The base engagement members 162, 163 can engage the cover engagement portions 142, 143 when the cover 160 is rotated into the closed position. For example and as embodied herein, base engagement members 162, 163 can be configured as one or more projections, and the cover engagement portions 142, 143 can be configured as one or more ramped surfaces sized and positioned to be engaged by the one or more projections. In operation, when rotating the cover 160 toward the closed position, base engagement members 162, 163 abut and move along the ramped surfaces of the cover engagement portions 142, 143. As such, the base engagement members 162, 163 can urge the movable syringe engagement element 140 disclosed herein along the longitudinal axis an increasing distance as the base engagement members 162, 163 move further along the ramped surfaces, thereby urging syringe engagement member 140, along with cap engagement portion 141, from the first position to the second position. Furthermore, the base engagement members 162, 163 can include lock members 164, 165. The lock members 164, 165 can abut or latch against the cover engagement portions 142, 143 when the cover 160 is in the closed position to secure the cover 160 in the closed position.

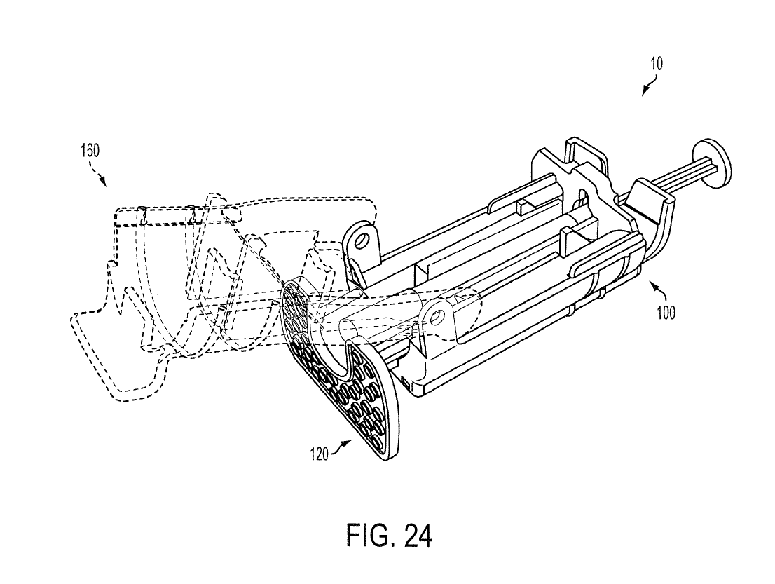

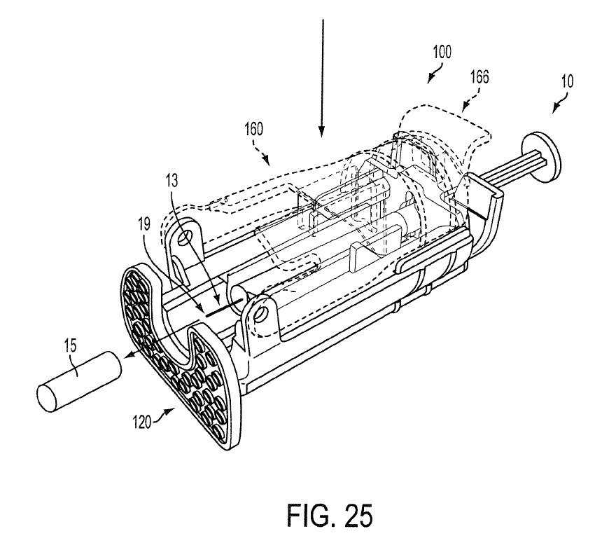

Referring now to FIGS. 23-26 for the purpose of illustration and not limitation, and with reference to a kit including a syringe and self-injection tool of the disclosed subject matter, operation of the self-injection tool 100 is shown and described. It is understood that the kit disclosed herein can include some or all of the features described in detail above. As depicted in FIG. 22, a flange adaptor 170, if present, can be mounted on the flange of the syringe. The Syringe 10 is then positioned within the barrel engagement portion 112, the cap engagement portion 141 disposed between the shoulder 12 of the syringe 10 to engage end 21 of the cap 15, as depicted in FIGS. 23 and 24. As discussed herein, when the cover 160 is rotated from the open position to the closed position, the base engagement members 162, 163 engage cover engagement portions 142, 143 and urge the syringe engagement member 140, along with cap engagement portion 141, from the first position to the second position. As such, during rotation of the cover 160 from the open position to the closed position and movement of the syringe engagement member 140, cap engagement portion 141 moves relative the one or more flange engagement portions 152 along the longitudinal axis x_{b} to urge the cap 15 away from the barrel 11, as depicted in FIG. 25. However, because the shroud member 120 is biased toward the extended position, the needle 13 remains concealed from view.

As embodied herein, with the syringe 10 within the self-injection tool 100 and the cover 160 rotated to the closed position, the user can then perform an injection with the aid of the self-injection tool 100. In operation, the user can place the self-injection tool over a desired injection site, such that the planar engagement surface 124 of the distal end 122 of the shroud member 120 rests on the skin of the patient, with the needle directly above the injection site and the needle aligned perpendicular to the face of the shroud. In this position, the distal end 122 of the shroud 120 extends the extended distance D₂ from the distal end of the base 111, which is at least equal to or greater than D₁, the distance between the distal end 19 of the needle 13 and the distal end of the base 111. In this position, the needle is not yet in contact with the patient's skin. Additionally, the shroud member 120 can conceal the needle 13 from the user's view. The user can then push down on the self-injection tool 100, causing the shroud member 120 to move from the fully-extended position to the fully-retracted position. In the fully-retracted position, the distal end 122 of the shroud member 120 is a retracted distance D₃ from the distal end of the base 111, as depicted in FIG. 26. Accordingly, as the user presses the self-injection tool 100 against the injection site, the needle 13 moves past the shroud member 120 and is injected into the injection site. The user can then depress the plunger 16 to expel the beneficial agent through the needle 13 and into the injection site.

After injection, the user can pull the self-injection tool 100 away from the injection site to remove the needle 13 from the injection site. As the self-injection tool 100 is removed, the biasing member 23 can cause shroud member 120 to move back to the first or extended position. Accordingly, as the tool 100 is pulled away from the user, the needle 13 is again concealed by the shroud member 120 and concealed from the user's view. Additionally, this arrangement can protect against inadvertent needle sticks after use of the syringe 10, particularly if the shroud includes a tubular portion configured to fully surround the needle in the extended position. The shroud member 120 can also conceal the needle 13 from the user's view. In alternative embodiments, however, the shroud member 120 can be configured to remain locked in the second position until the cover 160 is opened, as described herein below.

For example, if desired, the cover 160 can be configured to create a friction engagement between the shroud member 120 and the base 110, such as the barrel engagement portions, to inhibit movement of the shroud until the cover is moved to the open position.

Alternatively, in other embodiments the proximal end 121 can further include locking projections 126. The lock mechanism 150 can be movable between a non-engaging position (when the syringe engagement element 140 is in the first position) and an engaging position (when the syringe engagement element 140 is in the second position). The locking projection 126 can be configured to engage the lock mechanism 150 when the lock mechanism 150 is in the engaging position and the shroud member is in the retracted position. The lock mechanism 150 can be movable to the non-engaging position and configured to disengage the locking projections 126 when the cover 160 is rotated relative the base from the closed position to the open position.

After use, the cover 160 can be moved back toward the open position by grip member 166. The lock members 164, 165 can thus be disengaged from the cover engagement portions 142, 143 by rotating the cover 160 away from the base 110. As the cover 160 rotates toward the open position, the syringe engagement member 140 can move back to the first position. The syringe 10 can be removed from the self-injection tool 100 and discarded appropriately. The self-injection tool 100 can thus be reused by inserting a new syringe 10.

The self-injection tool of the disclosed subject matter can be used for injection or delivery of any of a variety of suitable liquid substances of corresponding volume or dose. For example, suitable liquid substances can include any suitable nutritional, pharmaceutical, or biological agents. Suitable liquid substances can include a liquid beneficial agent, which can include a protein. In some embodiments, the liquid beneficial agent can include a TNF inhibitor or the like, such as adalimumab.

The self-injection tool can be made of any suitable medical device materials, including, but not limited to, plastic or other known materials. The device can be formed through any suitable technique, for example, but not limitation, injection molding.

While the disclosed subject matter is described herein in terms of certain preferred embodiments, those skilled in the art will recognize that various modifications and improvements can be made to the disclosed subject matter without departing from the scope thereof. Moreover, although individual features of one embodiment of the disclosed subject matter can be discussed herein or shown in the drawings of the one embodiment and not in other embodiments, it should be apparent that individual features of one embodiment can be combined with one or more features of another embodiment or features from a plurality of embodiments.

In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other manners within the scope of the disclosed subject matter such that the disclosed subject matter should be recognized as also specifically directed to other embodiments having any other possible combinations. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents.

1. A self-injection tool to assist with injecting a beneficial agent from a syringe, the syringe comprising a barrel having a first end and a second end, a shoulder proximate the first end, a needle in fluid communication with the barrel and having a proximal end and a distal end extending from the first end of the barrel, a flange disposed proximate the second end of the barrel, a cap covering the needle and having an end disposed proximate the shoulder, and a plunger movable within the barrel to expel beneficial agent through the needle, the self-injection tool comprising:
a base having a proximal end, a distal end, and a barrel engagement portion defining a cavity having a longitudinal axis and disposed between the proximal and distal end of the base to receive at least a portion of the barrel with the distal end of the needle extending a first distance from the distal end of the base; and
a shroud member having a proximal end and a distal end, the proximal end of the shroud member movably coupled to the base, the shroud member being movable between a fully-extended position wherein the distal end of the shroud member is an extended distance from the distal end of the base and a fully-retracted position wherein the distal end of the shroud member is a retracted distance from the distal end of the base;
wherein the extended distance is greater than or equal to the first distance, and the retracted distance is less than the first distance.

2. The self-injection tool of claim 1, wherein the shroud member is biased toward the extended position.

3. The self-injection tool of claim 1, wherein the distal end of the shroud member comprises a substantially planar engagement surface having a notch therein.

4. The self-injection tool of claim 1, wherein the distal end of the shroud member comprises a substantially planar engagement surface having a through-hole therein.

5. The self-injection tool of claim 1, wherein the shroud member further comprises a tubular portion disposed between the proximal end and the distal end of the shroud member.

6. The self-injection tool of claim 1, wherein the base further comprises at least one flange engagement portion defining a slot to receive at least a portion of the flange and inhibit movement of the barrel along the longitudinal axis.

7. The self-injection tool of claim 6, wherein the base includes a housing, and wherein the self-injection tool further comprises a cover hingedly joined to the base and rotatable relative the base between an open position and a closed position.

8. The self-injection tool of claim 7, wherein the barrel engagement portion is movable relative the housing, the barrel engagement portion further comprising a cap engagement portion having an opening sized to receive at least a portion of the first end of the barrel, the cap engagement portion positioned to engage the end of the cap proximate the shoulder, the cap engagement portion being movable relative the housing to urge the cap away from the barrel.

9. The self-injection tool of claim 8, wherein the cap engagement portion comprises a U-shaped notch.

10. The self-injection tool of claim 7, wherein the housing comprises a bottom and at least one sidewall extending from the bottom, and wherein the cover is joined to the housing proximate at least one of the at least one sidewall.

11. The self-injection tool of claim 10, wherein the at least one sidewall has a substantially rounded shape.

12. The self-injection tool of claim 10, wherein the housing comprises a proximal face extending from the bottom and disposed perpendicular to the at least one sidewall.

13. The self-injection tool of claim 12, wherein the proximal face comprises an opening to receive at least a portion of the plunger therethrough when the at least a portion of the barrel is disposed within the barrel engagement cavity.

14. The self-injection tool of claim 7, wherein the cover has at least one projection to engage the barrel when the cover is in the closed position.

15. The self-injection tool of claim 8, wherein the cap engagement portion is movable relative the at least one flange engagement portion when the cover is rotated relative the base from the open position to the closed position.

16. The self-injection tool of claim 7, wherein the syringe is held in engagement when the cover is moved to the closed position.

17. The self-injection tool of claim 6, further comprising a flange adaptor having a recess to receive the flange and at least one wing element configured to be engaged by the flange engagement portions.

18. The self-injection tool of claim 7, wherein the base further comprises a lock mechanism movable between a non-locking position and a locking position when the cover is rotated relative the base from the open position to the closed position;
the proximal end of the shroud comprises at least one locking projection; and
wherein the at least one locking projection is configured to engage the lock mechanism when the lock mechanism is in the locking position and the shroud member is in the retracted position.

19. The self-injection tool of claim 18, wherein the lock mechanism is movable from the locking position toward the non-locking position to disengage the at least one locking projection when the cover is rotated relative the base from the closed position toward the open position.

20. The self-injection tool of claim 1, wherein the beneficial agent comprises TNF inhibitor.

21. A kit comprising:
a syringe comprising a barrel having a first end and a second end and defining a longitudinal axis therebetween, a shoulder proximate the first end, a needle in fluid communication with the barrel and having a proximal end and a distal end extending from the first end of the barrel, a flange disposed proximate the second end of the barrel, a cap covering the needle and having an end disposed proximate the shoulder, and a plunger movable within the barrel to expel beneficial agent through the needle; and
a self-injection tool comprising a base having a proximal end, a distal end, and a barrel engagement portion defining a cavity disposed between the proximal and distal end of the base to receive at least a portion of the barrel with the distal end of the needle extending a first distance from the distal end of the base;
a shroud member having a proximal end and a distal end, the proximal end of the shroud member movably coupled to the base, the shroud member being movable between a fully-extended position wherein the distal end of the shroud member is an extended distance from the distal end of the base and a fully-retracted position wherein the distal end of the shroud member is a retracted distance from the distal end of the base;
wherein the extended distance is greater than or equal to the first distance, and the retracted distance is less than the first distance.
